(19) United States
(12) Patent Application Publication
(10) Pub. No.: US 2019/0117143 A1
(43) Pub. Date: Apr. 25, 2019

(54) Methods and Apparatus for Assessing Depression

(71) Applicants: Massachusetts Institute of Technology, Cambridge, MA (US); The General Hospital Corporation, Boston, MA (US)

(72) Inventors: Szymon Fedor, Boston, MA (US); Asma Ghandeharioun, Cambridge, MA (US); Rosalind Picard, Newton, MA (US); Dawn Ionescu, Encinitas, CA (US)

(21) Appl. No.: 16/168,378

(22) Filed: Oct. 23, 2018

Related U.S. Application Data
(60) Provisional application No. 62/576,040, filed on Oct. 23, 2017.

(51) Int. Cl. A61B 5/16; A61B 5/0205

(52) U.S. Cl. CPC A61B 5/165; A61B 5/02405; A61B 5/0205

(57) ABSTRACT

An automated system may estimate a patient's level of depression throughout a monitoring period. The system may do so without ever receiving any self-reports from the patient, such as patient answers to a survey regarding the patient's affect. The system may predict the patient's depression level based on passive sensor data regarding the patient during the monitoring period. The passive sensor data may include physiological measurements, such as electrodermal activity measurements and accelerometer measurements. The passive data may also comprise data regarding the patient's smartphone and texting usage. The system's predictions may also be based on a single depression rating for the patient by a clinician, without any further assessments by the clinician.

### RELATED APPLICATIONS

This application claims the benefit of U.S. Provisional Application No. 62/576,040 filed Oct. 23, 2017 (the “Provisional”).

### FIELD OF TECHNOLOGY

The present invention relates generally to health technology.

### COMPUTER PROGRAM LISTING

The following 23 computer program files are incorporated by reference herein: (1) dimensionality_reduction.txt with a size of about 6 KB; (2) app_features.txt with a size of about 3 KB; (3) call_features.txt with a size of about 4 KB; (4) combine_features.txt with a size of about 2 KB; (5) display_features.txt with a size of about 3 KB; (6) EDA_features.txt with a size of about 1 KB; (7) eda_features_calculation.txt with a size of about 62 KB; (8) EDA_motionless_features.txt with a size of about 1 KB; (9) HRV_features .txt with a size of about 1 KB; (10) location_features.txt with a size of about 4 KB; (11) location_smart_features.txt with a size of about 6 KB; (12) motion_features.txt with a size of about 2 KB; (13) sleep_features.txt with a size of about 1 KB; (14) sms_features.txt with a size of about 4 KB; (15) survey_features.txt with a size of about 14 KB; (16) survey_label_individualization.txt with a size of about 2 KB; (17) HDRS_imputation_survey.txt with a size of about 16 KB; (18) basic.txt with a size of about 11 KB; (19) boosting.txt with a size of about 14 KB; (20) ensemble.txt with a size of about 11 KB; (21) gp.txt with a size of about 10 KB; (22) rf.txt with a size of about 13 KB; and (23) robust.txt with a size of about 11 KB. Each of these 23 files were created as an ASCII .txt file on Oct. 17, 2018.

### SUMMARY

In illustrative implementations of this invention, an automated system estimates a patient's level of depression during a monitoring period. The system may do so: (a) without ever receiving self-reports from the patient, such as patient answers to a survey regarding the patient's affect; and (b) with only a single assessment by a clinician of the patient's level of depression.

In illustrative implementations, the system predicts the patient's depression level during a monitoring period, based on only two types of data. First, the system may rely on passive sensor data regarding the patient. The passive sensor data may include physiological measurements taken during the monitoring period, such as electrodermal activity measurements, accelerometer or other motion measurements, or measurements of temperature or heart rate. The passive data may also include data regarding smartphone usage and SMS usage by the patient during the monitoring period. Second, the system may also use a single depression rating for the patient. For instance, this single depression rating may made by the clinician at the start of the monitoring period or later in the monitoring period. Any type of smart device with cellular phone functionality may be employed as a “smartphone” for this purpose, including a smartwatch, smartglasses or a conventional smartphone.

Thus, the automated system of the present invention solves a technical problem with conventional methods of automatically assessing depression. Conventional methods tend to rely heavily on patient self-reports, such as patient answers to surveys regarding their emotional state. Unfortunately, patient self-reports are tedious for a patient to fill out, and tend to be unreliable—in part because patients do not fill out some of the self-reports or answer them too quickly (in order to avoid the tedium of answering carefully). In contrast, the present invention solves this problem—because it may estimate a patient's depression level without ever receiving a patient self-report.

In illustrative implementations, the automated system estimates or predicts a user's level of depression. This estimate or prediction may be quantified. Likewise, this estimate or prediction may be expressed in terms of any depression rating scale. For instance, in some cases, the estimate or prediction is expressed in terms of the Hamilton Depression Rating Scale (HDRS). For example, in HDRS: (a) a rating between 0 and 7 is normal; (b) a rating between 8 and 13 indicates mild depression; (c) a rating between 14 and 18 indicates moderate depression; (d) a rating between 19 and 22 indicates severe depression; and (e) a rating greater than 23 indicates very severe depression. Alternatively, any other depression rating scale may be employed, such as the Beck Hopelessness Scale, CES-D (Centre for Epidemiological Studies—Depression Scale), CES-DC (Centre for Epidemiological Studies Depression Scale for Children), EPDS (Edinburgh Postnatal Depression Scale), GDS (Geriatric Depression Scale), Hospital Anxiety and Depression Scale, KADS (Kutcher Adolescent Depression Scale), MADRS (Montgomery-Asberg Depression Scale), WSAS (Weinberg Screen Affective Scale), PHQ-9 or any other depression rating scale.

In illustrative implementations, the automated system includes a machine learning algorithm (e.g., an ensemble machine learning model) that is trained on a training dataset. The training dataset may include more types of data than the dataset that is used by the trained system to make predictions.

Specifically, in some cases, the training dataset includes five types of data for each patient in a set of multiple patients. First, the training dataset includes, for each training patient, periodic (e.g., bi-weekly) depression ratings by a clinician during the training period. Second, the training dataset includes, for each training patient, imputed depression ratings for times (e.g., days) that are in between the clinician ratings. The imputed depression ratings may be estimated based on patient self-reports during the training period. Thus, in illustrative implementations: (a) patient self-reports are gathered from multiple patients during training; but (b) once the system is trained, the system may estimate a new patient's depression level without receiving any patient self-report from the new patient. Third, fourth and fifth, the training dataset may include, for each of the training patients, physiological sensor data, smartphone usage data and SMS data gathered during the training period.

Thus, in illustrative implementations of this invention, another technical problem is solved. The problem is that it may be difficult, time-consuming and expensive to gather depression ratings by clinicians to create a training dataset. In illustrative implementations of this invention, this problem is solved by gathering a relatively small number of depression ratings by clinicians for the training dataset. To add more datapoints for the training dataset, the system may impute depression ratings for multiple times between the clinician depression ratings. These imputed depression ratings (e.g., imputed HDRS depression ratings) may be computed based on patient self-reports gathered during the training period.

Here is an example that includes acquiring the training dataset, training on it, and then using the trained system to estimate a depression rating for a patient. In this example: During a training period, the automated system accepts, as input, depression ratings by a clinician (e.g., bi-weekly HDRS ratings by clinicians) for multiple patients. During the training period, the automated system accepts, as input, self-reports by the patients (e.g., answers to surveys, multiple times daily). During the training period, the system gathers physiological data (e.g., EDA, SCR, accelerometer, or other motion data), SMS usage data (e.g., number of incoming and outgoing texts) and smartphone usage data (e.g., number of outgoing, incoming and missed calls, duration of calls, whether display is on or off, GPS data, and app usage). Based on the patient self-reports, the system estimates depression ratings for missing datapoints during the training period (which datapoints correspond to times between the depression ratings by clinicians). The system creates an enlarged dataset of depression ratings for the training period, comprising the ratings by clinicians and the ratings estimated from the patient self-reports. A machine learning program is trained on a training dataset, which training dataset comprises: (1) the enlarged dataset of depression ratings for the training period; and (2) the physiological data, SMS usage data and smartphone usage data gathered during the training period. After the training (e.g., at the start of a monitoring period or later), the system accepts, as input, a depression rating by a clinician regarding a new patient. During the monitoring period, the system gathers passive data regarding the new patient, which passive data comprises the same type of physiological data, SMS usage data, and smartphone usage data. The system employs the trained ensemble model to estimate, based on this passive data and the clinician depression rating, one or more depression ratings for the new patient (e.g., a depression rating for each of multiple dates during the monitoring period other than the date of the clinician depression rating). Alternatively, the new patient may be one of the patients used for training. The example described in this paragraph is non-limiting. This invention may be implemented in many other ways.

In illustrative implementations, a clinician who makes a depression rating for a patient may be, for instance, a psychologist, psychiatrist, primary care doctor, nurse or other health care worker, or may be any person with expertise in psychology or psychiatry. In some cases, a clinician makes a depression rating for a patient based on observations of the patient during a face-to-face meeting with the patient or during a remote interview with the patient (e.g. via an audiovisual communication system).

In some implementations of this invention, machine learning techniques are applied to objective data that is captured passively and continuously from E4 wearable wristbands and from sensors in an Android® phone. The machine learning models may be employed to predict a patient's level of depression on the Hamilton Depression Rating Scale (HDRS). Input data may include electrodermal activity (EDA), sleep behavior, motion, phone-based communication, location changes, and phone usage patterns. In some implementations: (a) the input data is processed by a feature generation and transformation process, (b) missing clinical scores are imputed from self-reported measures; and (c) depression severity is predicted from continuous sensor measurements.

In some implementations of this invention, at least the following features are tracked: sleep, motion, incoming messages, variability in location patterns, and symmetry/asymmetry of EDA between the right and the left wrists. This is because depression may be positively correlated with more irregular sleep, less motion, fewer incoming messages, less variability in location patterns, and higher asymmetry of EDA between the right and the left wrists.

In experimental tests, a prototype of this invention achieved accurate depression ratings. For instance, a prototype of this invention calculated imputed HDRS ratings with a 2.8 RMSE (root mean squire error) and predicted HDRS ratings with a 4.5 RMSE. These RMSE errors are quite low, relative to the complete range of HDRS (0-52).

The Summary and Abstract sections and the title of this document: (a) do not limit this invention; (b) are intended only to give a general introduction to some illustrative implementations of this invention; (c) do not describe all of the details of this invention; and (d) merely describe non-limiting examples of this invention. This invention may be implemented in many other ways. Likewise, the Field of Technology section is not limiting; instead it identifies, in a general, non-exclusive manner, a field of technology to which some implementations of this invention generally relate.

### BRIEF DESCRIPTION OF THE DRAWINGS

FIG. 1 is a flowchart for a method of rating depression.

FIGS. 2A, 2B and 2C together are a flowchart for a method of rating depression.

FIGS. 3A and 3B show a physiological sensor.

FIG. 4 shows hardware for a system of monitoring depression.

The above Figures are not necessarily drawn to scale. The above Figures show some illustrative implementations of this invention, or provide information that relates to those implementations. The examples shown in the above Figures do not limit this invention. This invention may be implemented in many other ways.

### DETAILED DESCRIPTION

The following 46 paragraphs describe a prototype of this invention. The prototype is a non-limiting example of this invention. This invention may be implemented in many different ways.

### Prototype—General

The prototype estimates, based on passive sensing, depressive symptoms as rated on the Hamilton Depression Rating Scale (HDRS). The prototype utilizes data from Empatica® E4 wearable sensors and embedded sensors within an Android® smartphone.

In the prototype, HDRS is captured bi-weekly by a clinician, as part of the clinician's standard practice. A two-step prediction process is employed: First, a surrogate (self-reported data) is employed to predict HDRS and to impute the missing HDRS values (from the dates when the HDRS was not assessed by a clinician) to construct an increased dataset “HDRS-I”. Second, passive phone and wearable sensor measures are used for predicting the HDRS-I values.

In the prototype, depressive symptoms are clinically assessed by using a Hamilton Depression Rating Scale (HDRS) as scored by an expert clinician in a patient interview. For each patient, the clinical form of HDRS data is collected bi-weekly in a face-to-face meeting between a clinician and the patient. For each patient, the patient's depression level for the remaining dates is estimated by using machine learning that incorporates daily patient self-reports.

In the prototype, progressive change of symptoms is measured in order to enable just-in-time interventions.

In a test of the prototype, twelve patients diagnosed with MDD (major depressive disorder) from Massachusetts completed an 8-week protocol. Participants included 9 females and 3 males from white, Hispanic, African-American, and Asian races and aged between 20 and 73 years old (mean=37, std=17). The protocol involved tracking depressive symptoms and mobile phone usage. Movisens™ was used to measure incoming and outgoing text messages and phone calls, location, app usage, and screen on/off behavior. Patients also wore Empatica® E4 wristbands that recorded accelerometer data and electrodermal activity 23 hours a day. Measurements were processed to obtain daily aggregate measures. Participants were clinically assessed for depression symptoms biweekly using the HDRS. Table 1 summarizes the number of observations for each modality.

TABLE 1
Dataset summary after computing daily features.

| Modality | # of Datapoints |
|---|---|
| Physiological signals | 540 |
| Phone passive usage data | 605 |
| Interactive surveys | 503 |
| Clinical measures | 59 |

### Prototype—Physiological Data

In the prototype, E4 sensors worn on each wrist capture continuous electrodermal activity (EDA) via the measurement of skin conductance (4 Hz sampling rate), temperature (4 Hz sampling rate), and 3-axis accelerometer data (32 Hz sampling rate). This data is measured in 6-hour intervals, labeled as morning, afternoon, evening, and night. The 6-hour interval provides a balance between granularity and ratio of missing values. Aggregate daily measures are also calculated. In some cases, many of the features that are employed by the prototype to impute or predict HDRS are calculated for all these intervals. However, some other features that are employed by the prototype to impute or predict HDRS (e.g., night sleep onset time, sleep duration, or other sleep features) are not calculated for each of these 6-hour intervals.

In the prototype, the EDA signal is filtered out when the corresponding skin temperature is below 31° C. to exclude the measurements when the sensor is not worn. Then a 6th order Butterworth low-pass filter (1 Hz cutoff frequency) is applied. The prototype computes: (a) EDA and the fraction of time the sensor is recording the signal; and (b) the number of skin conductance response (SCR) peaks and their average amplitude. Measurements of SCR peaks and EDA are advantageous, because skin conductance level may distinguish between depressed and healthy individuals.

The prototype computes the following measures of asymmetry of EDA at different wrists: (a) difference between average EDA value; (b) difference between number of SCRs; and (c) difference between SCL (skin conductance level) and SCR signals using Convex Optimization Approach. Recording asymmetry in EDA is advantageous, because asymmetry in EDA between the wrists may provide affective information.

The prototype applies a 5th order Butterworth low-pass filter (10 Hz cutoff frequency) to the accelerometer data. The output is then translated into motion features by calculating the vector magnitude VM of the z-axis acceleration data using the following formula:

VM=Σₜ₌₀^{N}VMₜ+|R_{(z,t)}−M_{z}| (Eq. 1)

where R_{(z,t)} the raw accelerometer z-axis sample, M_{z} is the running mean in a 5-second window of the z-axis signal, and N is the number of raw data samples received in one second.

The prototype also calculates average, median, and standard deviation of motion for the mentioned time intervals as well as the fraction of time in motion. The prototype also keeps metadata (such as the fraction of time within the time interval that the data were not missing).

The prototype also calculates objective sleep based on accelerometer data for 30 second epochs using an ESS (Epworth sleepiness scale) method. The prototype calculates sleep duration, sleep onset time (time elapsed since noon), maximum duration of uninterrupted sleep, number of wake-ups during the night, and the time of waking up (time elapsed since midnight). The prototype also computes a sleep regularity index (SRI):

$$SRI = ( \frac{1}{2} ) 1 + \frac{1}{( T - τ )} {\int }_{0}^{T τ} s ( t ) s ( t + τ ) dt \qquad ( Eq . 2 )$$

where data were collected for y=[0,T], τ=24, s(t)=1 during sleep and s(t)=−1 during wake. The SRI ranges between 0 (highly irregular sleep) and 1 (consistent sleep every night). The prototype also gathers meta-data such as the fraction of time that data is recorded over nighttime (between 8 pm-9 am) as well as over the period of 24 hours.

### Prototype—Passive Phone Usage Data

In the prototype, Movisens™ on Android® collects measures of how the participant is using his or her mobile phone and how s/he is interacting with other people using the mobile phone. More specifically, the prototype captures meta-data of calls, text messages, app usage, display on/off behavior, and location. Passive data is captured 24/7 (i.e., throughout each 24 hour day). The content of the calls/texts, actual phone numbers, websites visited, and the content of the applications is not collected.

The prototype employs 3-hour intervals for the passive phone data. For example, 6 am-9 am represents early morning while 9 pm-12 am corresponds to late evening. The prototype also calculates aggregate daily measures.

To quantify call data, the prototype calculates the number of incoming, outgoing, and missed calls daily and over the 3-hour periods within the day. In a similar manner, the prototype calculates mean, median, and standard deviation (SD) of the duration of incoming, and outgoing calls. Also, the prototype calculates the incoming/outgoing ratio both for the number of calls and the duration of calls on a daily basis.

To quantify SMS data, a similar approach is employed. The prototype calculates the number of incoming and outgoing texts daily and over 3-hour periods within the day. The prototype also calculates a daily incoming/outgoing ratio of the number of text messages received or sent respectively.

Turning the display on/off is also an indication of phone usage. Thus, the prototype computes the mean, median, and SD of duration of screen-on within the mentioned intervals. The prototype also calculates the number of the times the screen has been turned on over these periods. Note that these two correspond to different behaviors; Long screen-on duration is related to actively using the phone while a great number of screen-ons is related to consistently checking the phone which might be a sign of anxiety or anticipation.

For location data, the prototype calculates mean, median, and SD of latitude and longitude along with the number of data points that have been captured for each time period. The prototype calculates total location mean, median, and SD by averaging values from latitude and longitude.

For app usage, the prototype encodes the app category using the following list: game, email, web, calendar, communication, social, maps, video streaming, photo, shopping, and clock. Then, the prototype calculates the total duration and the number of app category usage in the different mentioned time intervals.

The prototype employs a Movisens™ graphical user interface displayed on a mobile phone or computer screen, in order to administer short questionnaires about overall health condition, sleep, mood, stress, anxiety, alcohol/drugs/caffeine usage, and social interaction. These questionnaires may be completed each day upon awakening, at bedtime and twice during the day at random times.

For assessing mood, the prototype employs Positive and Negative Affect Schedule (PANAS), a scale for measuring affect. The 20 item questionnaire has been split into two 10-item questions that are administered twice during the day at random times.

The prototype preprocesses the data: the prototype adds how long it took the participant to fill in the survey and removes responses that took less than a second and are likely noise. This meta-data may also be informative; for example, long pauses while responding to surveys may represent motor slowing (a common symptom of depression), cognitive load, trouble remembering, or not being sure about the response. Short response time, on the other hand, may represent trivial answers or not reading through the questions. The prototype calculates total alcohol (standard drink measure) and caffeine consumption (milligram) by summing the relevant features from the survey. The prototype converts categorical features to their one-hot representation. The prototype also labels data by day of the week. Including day of the week is advantageous, because it has been shown to influence the aggregate number of smiles which can be an indication of positive valence mood.

Since HDRS is closely related to self-reported mood, the prototype computes more detailed mood information. For instance, the prototype calculates total positive affect (PA) and negative affect (NA) on a daily basis by averaging responses to relevant survey questions. The prototype adds an average of the past week's PA and NA. The prototype adds a weighted average of PA and NA, when the effect of affect diminishes exponentially over time when going back in history, e.g., yesterday's mood is half as important as today's mood in the weighted average measure. The prototype calculates the NA/PA ratio for the daily, average weekly, and weighted average weekly measures. To capture mood oscillation, the prototype includes the standard deviation of mood on a weekly basis and for the duration of the relevant period (e.g., training period or monitoring period).

During each biweekly visit during training, participants are assessed by a clinician for depressive symptoms using the HDRS. HDRS is a standard test for quantifying depressive symptoms which ranges between 0 and 52. Table 2 summarizes the depression severity in relation to HDRS.

TABLE 2
HDRS values and levels of depression severity.

| HDRS | Depression Severity |
|---|---|
| 0-7 | Normal |
| 8-13 | Mild Depression |
| 14-18 | Moderate Depression |
| 19-22 | Severe Depression |
| ≥23 | Very Severe Depression |

### Prototype—Feature Transformation and Selection

In the prototype, combining the features results in over 700 features for a training dataset. Compared to the small number of data points (where each datapoint corresponds to a specific patient on a specific day), this number of features can easily result in over-fitting the model to the training set. One possibility is to use regularization tricks such as L1 to enforce selection of only a small number of features. However, for features that are non-linearly related, transforming the features into a new space through a non-linear transformation may be more beneficial. For example, several noisy measurements of a similar phenomena may not be informative on their own, but a transformed version of them may be a better predictor. Toward this end, the prototype may perform three different dimensionality reduction algorithms to reduce the dimensionality of the feature-set: (a) PCA (principal component analysis), (b) kernel PCA with radial-basis function kernel, and (c) truncated SVD (singular-value decomposition) methods. The prototype bounds the number of selected features while keeping as few features as possible to explain the variance of data.

The prototype creates 3 datasets: one including all features, one including daily features only, and one including the daily features and the features of the previous day. The prototype conducts the feature transformations on these three datasets.

### Prototype—HDRS Imputation Based on Survey Data

In an example of a dataset collected by the prototype, a strong positive correlation exists between average weekly negative/positive affect (M=0.86, SD=0.38) and HDRS scores (M=19.64, SD=7.60), r=0.70, p=0.00, n=44. Data points with missing mood reports from surveys have been removed from this dataset. This reduces the number of data points in this dataset from 59 available HDRS measurements to 44, where each datapoint corresponds to a specific patient on a specific day. The prototype utilizes survey data to estimate a “gold-standard measure”, HDRS-I, in between clinical assessments.

For this prototype, the input features include daily PA, NA, and NA/PA ratio. The prototype also computes average and standard deviation of these values over the past week and over the duration of the relevant period (e.g., training period or monitoring period). Also, the prototype calculates weekly weighted average of these values where the effect of affect diminishes exponentially over time. The prototype then imputes the missing values to construct a 10-times-larger dataset. Thus, the prototype may use two sets of models to predict the HDRS score from survey data: regularized regression and robust-to-outlier methods.

In the prototype, the regression methods (employed when imputing HDRS ratings for missing datapoints) include lasso, ridge, and elasticNet which use L1, L2, and a combination of the two as regularization metrics, respectively. Note that the L1 regularization term may act as a feature selection mechanism by pushing coefficients of most of the variables to be exactly zero, while L2 may push many coefficients to near zero values but does not remove them completely. The prototype also employs (when imputing HDRS ratings for missing datapoints) regression without regularization with the reduced and transformed features.

The prototype also employs (when imputing HDRS ratings for missing datapoints) Theil-Sen estimator, random sample consensus (RANSAC), and huber algorithms. These models have a built-in sampling procedure that allows a fraction of data points to be outliers and thus helps the prototype to be robust against outliers or errors in formulation of the model.

For validation, the prototype splits the data into 90% training and 10% testing. The prototype uses cross-validation on the training set to select the best model and use it for imputing missing HDRS values. For instance, the prototype may employ leave-one-out cross-validation.

### Prototype—HDRS Prediction Based on Sensor Data

After imputing HDRS scores, the new dataset HDRS-I is over 500 datapoints (where each datapoint corresponds to a specific patient on a specific day). In some cases, the prototype employs (for prediction of HDRS ratings) machine learning models that do not require enormous amounts of training data.

For instance, in some cases, the prototype runs a long short-term memory (LSTM) network on the dataset as well as an augmented version of it. For augmentation, the prototype adds x*0.01*SD_{f} to each feature f where x is a random number between −0.5 and 0.5 and SD_{f} is the standard deviation of the values for that feature.

In the prototype, self-reported affect measures are used only in the imputation phase (i.e., to impute HDRS values for missing datapoints, to increase the number of datapoints in the training dataset (where each datapoint corresponds to a specific patient on a specific day). In the prototype, the self-reported affect measures are not used for HDRS prediction after training (e.g., for predicting HDRS ratings for a new patient, using the prototype's trained machine learning program). Instead, in the prototype, the trained machine learning (ML) program takes as inputs only an initial HDRS rating by a clinician for a new patient and passive data for the new patient (e.g. physiological sensor data and data regarding smartphone usage and SMS usage), and, based on these inputs, outputs an HDRS rating for the new patient.

For HDRS prediction (similar to the imputation phase), the prototype uses lasso, ridge, elasticNet, and unregularized regression.

Also, for HDRS prediction (similar to the imputation phase), the prototype uses Theil-Sen, RANSAC, and huber methods. However, the prototype loops through a larger list of model parameters to optimize within each model. The prototypes uses these models (Theil-Sen, RANSAC, and huber) for subsets of the data or for a reduced version of the data, since these models may perform better on relatively small datasets.

The prototype also employs adaptive boosting (AdaBoost) and Gaussian boosting (e.g., to combine weak regressors sequentially to improve performance).

The prototype also employs random forest with different numbers of estimators. The random forest is an ensemble method with multiple decision trees.

The prototype also employs Gaussian Process with different regularization parameters and different numbers of restart points to model the data. This is advantageous, because natural phenomena often follow a Gaussian distribution.

TABLE 3
Best prediction model.

| | | | | RMSE | | Baseline | |
|---|---|---|---|---|---|---|---|
| Model Type | Model | Parameters | Dataset | Validation | Test | Average | Median |
| Regression | Regression | | Kernel PCA subset | 5.2 | 4.9 | 7.1 | 7.1 |
| Robust | Ransac | ms = 0.3 | Kernel PCA subset | 5.0 | 4.9 | 7.1 | 7.1 |
| Boosting | AdaBoost | n = 50, lr = 1 | Subset data | 5.5 | 4.6 | 7.1 | 7.1 |
| Random Forest | — | n = 15 | Subset data | 5.4 | 4.6 | 7.1 | 7.1 |
| Gaussian Process | — | o = 0.1, n = 5 | Kernel PCA subset | 5.3 | 5.5 | 7.1 | 7.1 |
| Overall Ensemble | | k = 1 | selected by individual models | 5.8 | 4.5 | 7.1 | 7.1 |

The prototype combines the results from these different regressors to get a more robust estimator. The ensemble method first finds a set of k nearest neighbors from the training set for each point. It then chooses the model that performs best on that set as the estimator for this point. The heuristic behind this method is that slight modifications in the feature set do not change the output drastically. Thus, if a classifier is working well on similar points, chances are it works well for the current point, as well. Looking at k nearest points as opposed to only the most similar point is for smoothing purposes. Note that as the points become higher dimensional, the distance between them becomes less meaningful in explaining similarity between the points. Thus, in some cases, the prototype uses only the first 5 reduced features based on kernel-PCA and creates a KD tree and finds the k nearest neighbors to the point at hand.

The prototype may also include ensemble methods that return the average or median of all classifiers as another smoothing method.

In real life, some depressed patients see a doctor and get clinical assessments at some point in their life. One major issue is a high relapse ratio and not being able to regularly visit the doctor to re-assess the improvement or worsening of depressive symptoms. In such cases, the prototype may be easily deployed in real life to passively monitor the patients after the diagnosis. Thus, the prototype may have at least some history for each user.

For validation, the prototype splits the HDRS-I dataset into 90% training and 10% hold-out testing. However, the test set is only chosen from the original HDRS values (rather than the imputed ones). The prototype further chooses the test set in such a way as to mimic the real-life deployment scenario: no data point from the first two weeks is selected as test data. The prototype uses 10-fold-cross-validation on the training set to select the best model and use it for predicting HDRS values.

### Prototype—Evaluation of Imputation of HDRS for Missing Datapoints

For the prototype, root mean squared error (RMSE) is the primary metric used to validate the imputation phase. Table 4 shows the selected best model based on having the lowest RMSE on the validation set. Then the RMSE on the hold-out test set for each model is reported. This model is ridge regression on the subset of mood features from the survey data, obtaining a test RMSE of 2.8. A baseline prediction of reporting the average or median HDRS score results in an RMSE of 6.8.

Looking more closely at test results for the prototype provides insights about how the mood features may correspond to the HDRS score. Consider the coefficients with the highest absolute values: The coefficient for weekly average positive affect is −9.3, confirming that reported positive affect is negatively associated with HDRS score. Another interesting observation is the −7.4 coefficient of standard deviation of positive mood in the previous week. Depression is usually accompanied by anhedonia, withdrawal, and loss of engagement, which result in consistent low positive mood. Thus, a normal variation in positive mood is negatively associated with HDRS score. At the same time, there is a positive association between the average weekly negative affect and the HDRS score, shown by a positive 2.8 coefficient.

### Prototype—Evaluation of Prediction Phase

The new prediction model may be validated using RMSE. Table 3 shows the best performing model in each category and the overall customized ensemble method. The test RMSE for the ensemble method is 4.5 while it is 7.1 for the average or median baseline prediction.

### Prototype—Conclusion

The prototype may continuously measure depressive symptoms using a method that takes as inputs only: (a) an initial HDRS rating by a clinician; and (b) passive data captured from built-in sensors of a regular Android® phone and Empatica® E4 wristbands, including measures of EDA, sleep patterns, motion, communication, location changes, and phone usage patterns. The prototype may use a combination of machine learning techniques. In a test, the prototype predicted the imputed Hamilton Depression Rating Scale (HDRS) values on a hold-out set, obtaining a low error rate of 4.5 RMSE. Moreover, a post-hoc statistical analysis showed that poor mental health is associated with more irregular sleep, less motion, fewer incoming messages, less variability in location patterns, and higher asymmetry of EDA between the right and left wrists.

The preceding 46 paragraphs describe a prototype of this invention. The prototype is a non-limiting example of this invention. This invention may be implemented in many different ways.

### More Details

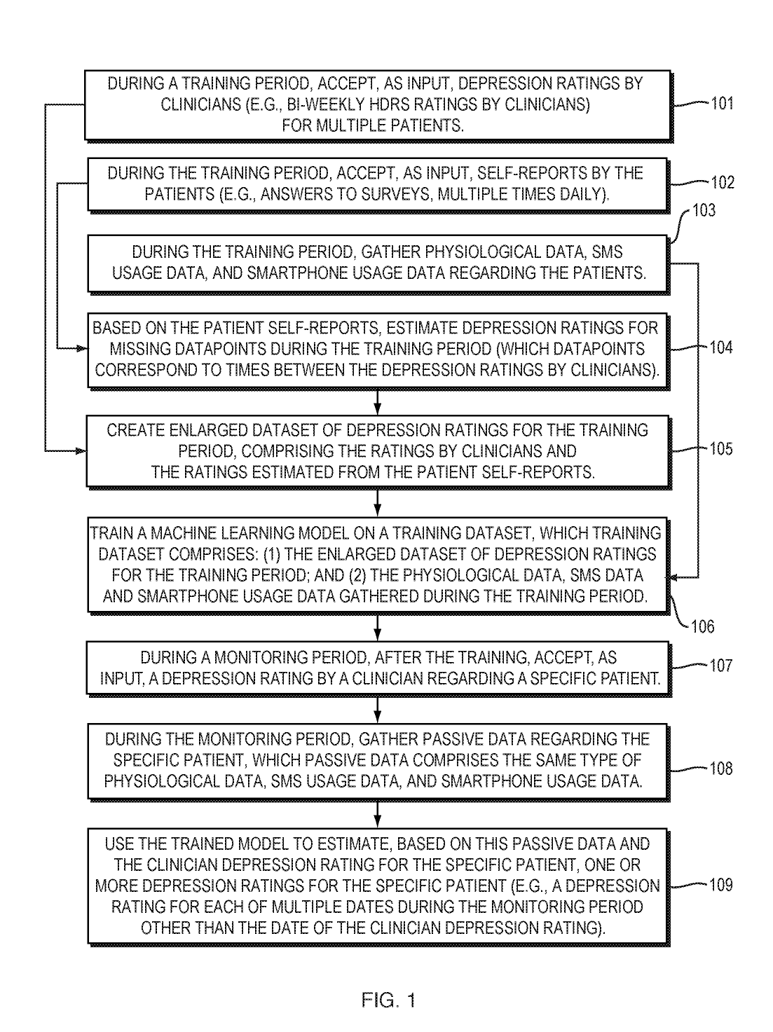

FIG. 1 is a flowchart for a method of rating depression, in an illustrative implementation of this invention. The method shown in FIG. 1 includes at least the following steps: During a training period, accept, as input, depression ratings by a clinician (e.g., bi-weekly HDRS ratings by clinicians) for multiple patients (Step 101). During the training period, accept, as input, self-reports by the patients (e.g., answers to surveys, multiple times daily) (Step 102). During the training period, gather physiological data, SMS usage data, and smartphone usage data regarding the patients (Step 103). Based on the patient self-reports, estimate depression ratings for missing datapoints during the training period (which datapoints correspond to times between the depression ratings by clinicians) (Step 104). Create an enlarged dataset of depression ratings for the training period, comprising the ratings by clinicians and the ratings estimated from the patient self-reports (Step 105). Train a machine learning program on a training dataset, which training dataset comprises: (1) the enlarged dataset of depression ratings for the training period; and (2) the physiological data, SMS data and smartphone usage data gathered during the training period (Step 106). During a monitoring period (after training), accept, as input, a depression rating by a clinician regarding a specific patient (Step 107). During the monitoring period, gather passive data regarding the specific patient, which passive data comprises the same type of physiological data, SMS usage data, and smartphone usage data (Step 108). Use the trained ensemble model to estimate, based on this passive data, one or more depression ratings for the patient (e.g., a depression rating for each of multiple dates during the monitoring period) (Step 109). In some cases, the machine learning program in Step 106 is an ensemble machine learning program. In Step 107, the depression rating by the clinician may be at the start of the monitoring period or later in the monitoring period.

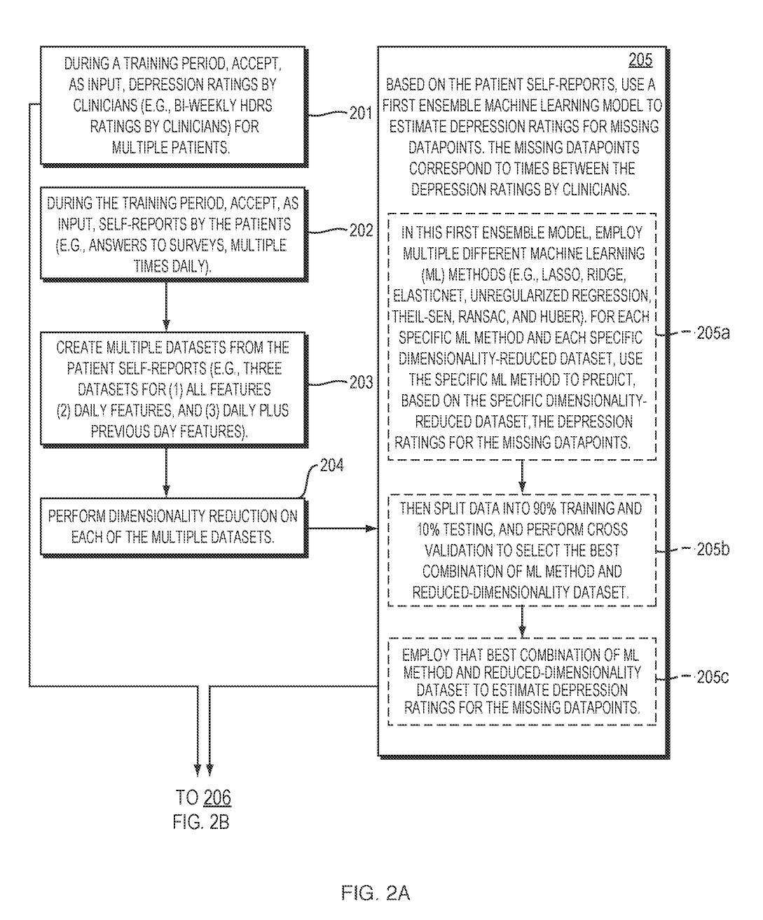

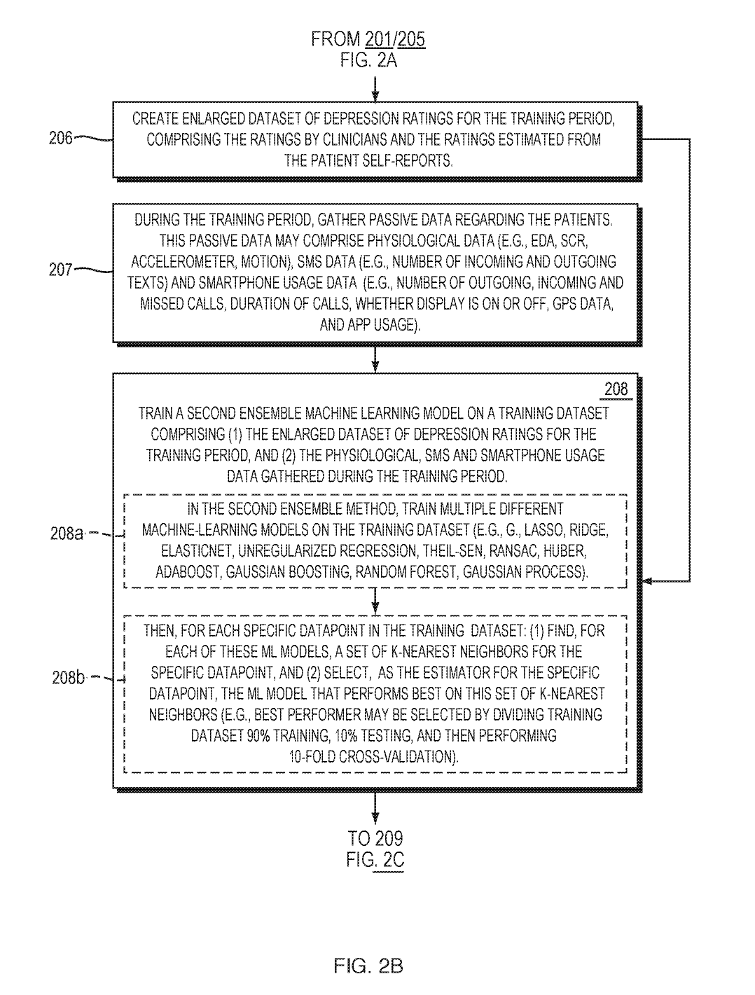

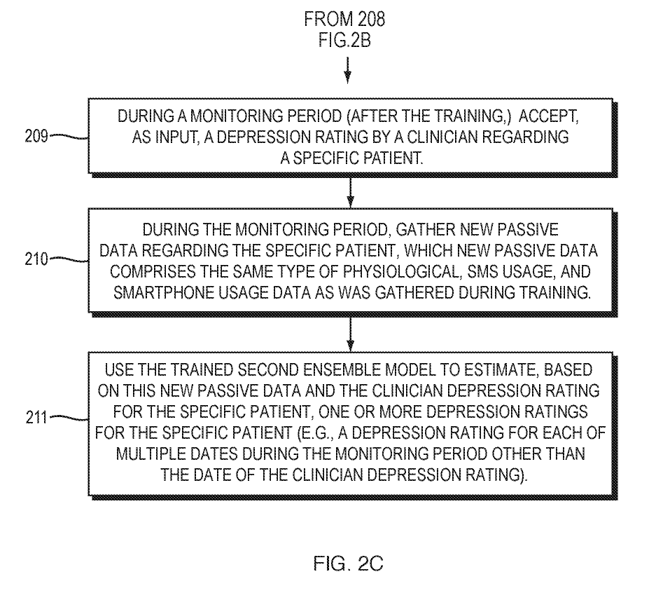

FIGS. 2A, 2B and 2C together are a flowchart for a method of rating depression, in an illustrative implementation of this invention. The method shown in FIGS. 2A, 2B and 2C includes at least the following steps: During a training period, accept, as input, depression ratings by clinicians (e.g., bi-weekly HDRS ratings by clinicians) for multiple patients (Step 201). During the training period, accept, as input, self-reports by the patients (e.g., answers to surveys, multiple times daily) (Step 202). Create multiple datasets from the patient self-reports (e.g., three datasets for (1) all features (2) daily features, and (3) daily plus previous day features) (Step 203). Perform dimensionality reduction on each of the multiple datasets (Step 204). Based on the patient self-reports, use a first ensemble machine learning model to estimate depression ratings for missing datapoints. The missing datapoints correspond to times between the depression ratings by clinicians (Step 205). Step 205 includes, as substeps, Steps 205a, 205b and 205c. In this first ensemble model, employ multiple different machine learning (ML) methods (e.g., lasso, ridge, elasticNet, unregularized regression, Theil-Sen, RANSAC, and huber). For each specific ML method and each specific dimensionality-reduced dataset, use the specific ML method to predict, based on the specific dimensionality-reduced dataset, the depression ratings for the missing datapoints (Step 205a). Then split data into 90% training and 10% testing, and perform cross validation to select the best combination of ML method and reduced-dimensionality dataset (Step 205b). Employ that best combination of ML method and reduced-dimensionality dataset to estimate depression ratings for the missing datapoints (Step 205c). Create an enlarged dataset of depression ratings for the training period, comprising the ratings by clinicians and the ratings estimated from the patient self-reports (Step 206). During the training period, gather passive data regarding the patients. This passive data may comprise physiological data (e.g., EDA, SCR, accelerometer, or other motion data), SMS usage data (e.g., number of incoming and outgoing texts) and smartphone usage data (e.g., number of outgoing, incoming and missed calls, duration of calls, whether display is on or off, GPS data, and app usage) (Step 207). Train a second ensemble machine learning model on a training dataset comprising (1) the enlarged dataset of depression ratings for the training period, and (2) the physiological, SMS and smartphone usage data gathered during the training period (Step 208). Step 208 includes, as substeps, Steps 208a and 208b. In the second ensemble method, train multiple different machine-learning models on the training dataset (e.g., g., lasso, ridge, elasticNet, unregularized regression, Theil-Sen, RANSAC, huber, AdaBoost, Gaussian boosting, random forest, Gaussian Process) (Step 208a). Then, for each specific datapoint in the training dataset: (1) find, for each of these ML models, a set of k-nearest neighbors for the specific datapoint, and (2) select, as the estimator for the specific datapoint, the ML model that performs best on this set of k-nearest neighbors (e.g., best performer may be selected by dividing training dataset 90% training, 10% testing, and then performing 10-fold cross-validation) (Step 208b). During a monitoring period (after training), accept, as input, a depression rating by a clinician regarding a specific patient (Step 209). During the monitoring period, gather new passive data regarding the specific patient, which new passive data comprises the same type of physiological, SMS usage, and smartphone usage data as was gathered during training (Step 210). Use the trained second ensemble model to estimate, based on the new passive data and on the clinician depression rating, one or more depression ratings for the patient (e.g., a depression rating for each of multiple dates during the monitoring period other than the date of the clinician depression rating) (Step 211). In Step 209, the depression rating by the clinician may be at the start of the monitoring period or later in the monitoring period.

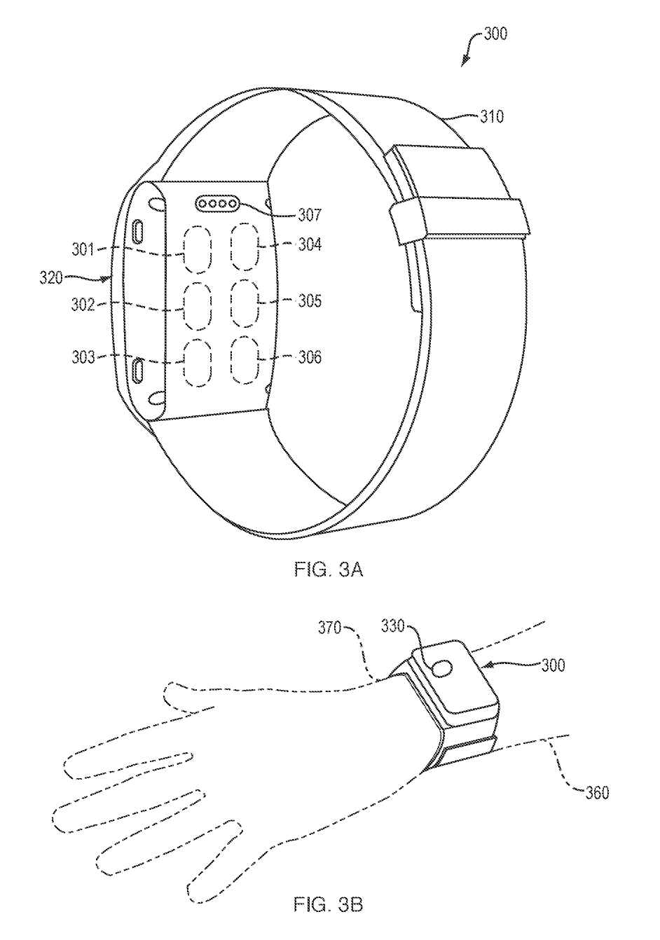

FIGS. 3A and 3B show a physiological sensor, in an illustrative implementation of this invention. In the example shown in FIGS. 3A and 3B, physiological sensor 300 is configured to be worn around a wrist 370 or around an arm 360 near a wrist. Physiological sensor 300 includes a sensor module 320, a wristband 310 and a USB port 307. Sensor module 320 houses a motion sensor 301, an EDA (electrodermal activity) sensor 302, a thermometer 303, a PPG (photoplethysmogram) sensor 304, an internal clock 305, a memory device 306, and an event marker button 330. In some cases, thermometer 303 comprises an infrared thermopile sensor. A human may press on button 330 to mark the time of an event (e.g., to cause the physiological sensor to associate contemporaneous sensor measurements with an event that occurred at that time). In some cases, motion sensor 301 comprises a 3-axis accelerometer, a 3-axis gyroscope, an inertial measurement unit, a magnetic motion sensor, or any optical, infrared or radio frequency motion sensor. The sensors (e.g., 301, 302, 303, 304) of the physiological sensor 300 may be positioned in any location in physiological sensor 300, not just the positions shown in FIGS. 3A and 3B. For instance, in some cases, EDA electrodes may be positioned on the inside of the wrist.

This invention is not limited to physiological sensors worn around or near a wrist. Alternatively, one or more physiological sensors (e.g., one or more of the sensors shown in FIGS. 3A and 3B) may be housed in one or more other wearable devices configured to be worn on other parts of a user's body, not just the wrist.

In FIGS. 3A and 3B, memory device 306 stores data that encodes measurements taken by the sensors (e.g., 301, 302, 303, 304). For instance, in some cases, memory device 306 stores up to 60 hours of measurements. In some cases, data encoding sensor measurements is exported via a USB port 330 and a USB cable to a wireless transmitter (e.g., a Bluetooth® or Wi-Fi® transmitter) or to a computer that is linked to the interne. Alternatively, the sensor data may be exported via any other wired connection or via a wireless module that is housed in physiological sensor 300.

In some cases, each physiological sensor (e.g., 300, 401, 402, 403) comprises an Empatica® E4 wristband sensor.

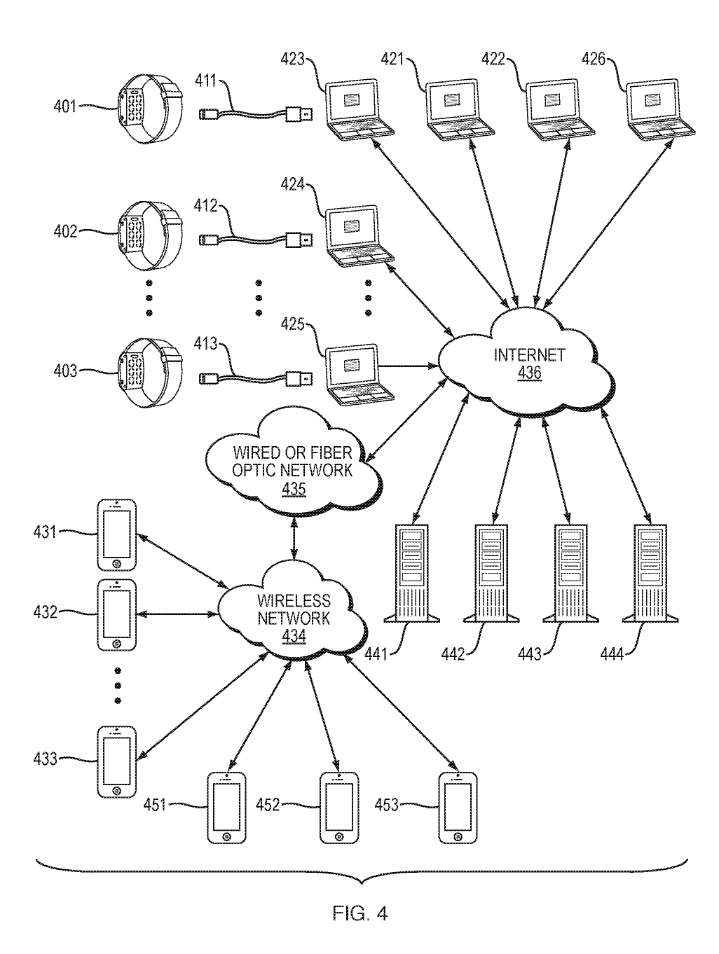

FIG. 4 shows hardware for a system of monitoring depression, in an illustrative implementation of this invention. In the example shown in FIG. 4, multiple patients each wear a physiological sensor (e.g., 401, 402, 403). For instance, each of these physiological sensors may be identical to sensor 300 shown in FIGS. 3A and 3B. Data encoding measurements taken by these physiological sensors may be sent from the physiological sensors to a server 441 via one or more wired, fiber optic or wireless communication networks. For instance, the physiological sensor data may be sent from physiological sensors to server 441 to server via USB cables (e.g., 411, 412, 413), computers (e.g., 423, 424, 425), and the internet 436. Server 441 may perform a program that tracks physiological data of users.

In FIG. 4, multiple patients may each have a smartphone (e.g., 431, 432, 433). Data regarding the patients' smartphone usage (e.g., number of outgoing, incoming and missed calls, duration of calls, whether display is on or off, GPS data, and app usage) may be collected. This data may be sent from the smartphones to a server 442 via one or more wired, fiber optic or wireless communication networks. For instance, this smartphone usage data may be sent to server 442 via a wireless network 434, wired or fiber optic network 435, and the internet 436. Server 442 may perform a program that tracks smart phone usage of users.

In FIG. 4, multiple patients may send and receive SMS messages via their smartphones (e.g., 431, 432, 433). Metadata regarding the patients' SMS usage (e.g., number of incoming and outgoing texts) may also be collected. Again, this metadata may be sent to a server 443 via one or more wired, fiber optic or wireless communication networks. Server 443 may perform a program that tracks SMS usage of users.

In FIG. 4, multiple patients may each enter self-reports (e.g., multiple times daily). Each self-report may comprise answers to survey questions. In some cases, the patients enter the self-reports via a graphical user interface displayed on display screens (such as the display screens of computers 423, 424, 425) or on display screens or touch screens of their smartphones (e.g., 431, 432, 433). These self-reports may be sent to server 444.

In FIG. 4, one or more clinicians enter depression ratings for multiple patients via one or more computers (e.g., 421, 422). These depression ratings may be sent via the internet 436 to server 444.

In FIG. 4, server 444 may request, from servers 441, 442, 443, data gathered during the training period regarding the multiple patients, and servers 441, 442, 443 may send this data to server 444.

Alternatively, there may only a single computer (e.g., 444) to which data regarding the patients during the training period (e.g. depression ratings, physiological data, smartphone usage data, SMS usage data) is sent.

In FIG. 4, server 444 may accept, as inputs: (a) depression ratings by clinician(s) for the multiple patients for each of multiple dates during a training period; and (b) the self-reports (multiple times daily) by the multiple patients during the training period. Based on these depression rating by clinicians and on these self-reports, server 444 may estimate depression ratings for each of the multiple patients for each of multiple intermediate times in the training period. These intermediate times may fall between the dates of the clinicians' depression ratings during the training period. For instance, in some cases: (a) a clinician inputs a bi-weekly depression rating for each patient (once every 14 days); and (b) server 444 estimates depression ratings for each patient for each other day in the training period.

In FIG. 4, server 444 trains a ensemble machine learning (ML) algorithm on a training dataset regarding the multiple patients. The training dataset may comprise the clinicians' depression ratings, the estimated depression ratings, the physiological sensor data, the smartphone usage data, and the SMS usage data, all acquired during the training period.

After the ensemble ML program is trained, it may evaluate a patient (the “user”) for depression. The user may be a new patient (whose data was not included in the training dataset). Alternatively, the user may be one of the patients whose data was used for training.

In FIG. 4, at the start of a monitoring period (after the training period), a clinician may input a depression rating for the user for a date after the training period. For instance, the clinician may input the depression rating via computer 421.

Also, during the monitoring period, physiological data regarding the user may be collected by a physiological sensor (e.g., 401). Likewise, during the monitoring period, the user may employ his or her smartphone (e.g., 431), including for phone calls, SMS messages and apps. Data regarding the user's smartphone usage and SMS usage during the monitoring period may be collected.

In FIG. 4, the clinician depression rating for the start of the monitoring period may be sent to server 444. The physiological data, smartphone usage data and SMS usage for the monitoring period may be sent to servers 441, 442, 443, respectively, and from there to server 444.

Alternatively, there may only a single computer (e.g., 444) to which data regarding the user during the monitoring period (e.g. depression ratings, physiological data, smartphone usage data, SMS usage data) is sent.

In FIG. 4, server 444 may compute a depression rating for the user for each of one or more times during the monitoring period. For instance, server 444 may perform calculations that involve the trained ML algorithm: (a) taking, as inputs, the clinician's depression rating for the user (at the start the monitoring period) and the physiological data, smartphone usage data, and SMS usage data for the user during the monitoring period; and (b) outputting a depression rating for the patient for a time (e.g., date) in the monitoring period.

In FIG. 4, after server 444 calculates a new depression rating for the user, server 444 may send a message regarding the new depression rating to the user, or to a health professional, or to a friend or family member of the user. For instance, the message may be sent by phone, by email, or by SMS message. In some cases, the message may include the new depression rating or may notify the recipient that a new depression rating may be accessed online. In some use scenarios, the message regarding the new depression rating is sent by phone call, by SMS message or by email and is received by the user's smartphone 451, or by a health professional's smartphone 452, or by a smartphone 453 of a friend or family member of the user. In some use scenarios, the message regarding the new depression rating is sent by email and is received by a computer of the user (e.g., 423), or by a health professional's computer 422, or by a computer 426 of a friend or family member of the user.

In some implementations, this invention comprises one or more computer that are programmed to perform one or more of the Computer Tasks (as defined herein).

In some implementations, this invention comprises one or more machine readable media, with instructions encoded thereon for one or more computers to perform one or more of the Computer Tasks.

In some implementations, this invention comprises participating in a download of software, where the software comprises instructions for one or more computers to perform one or more of the Computer Tasks. For instance, the participating may comprise (a) a computer providing the software during the download, or (b) a computer receiving the software during the download.

As discussed above, in some implementations, the system predicts a user's depression level based on passive data (e.g., physiological data, smartphone usage data and SMS usage data) gathered during a monitoring period and based on a clinician's assessment of the user's level of depression at the start of the monitoring period.

However, this invention is not limited to a scenario where the clinician's assessment of the user's level of depression occurs at the start of the monitoring period.

For instance, in some alternative implementations: (a) multiple models (e.g., N=20) are run in parallel, each taking an assumed initial HDRS rating (guess) across the full range where the model has been previously trained, and running all N models; and (b) when a depression rating for a patient by a clinician is received later, at a time after the start of the monitoring period, the system may choose the model(s) that were most accurate for that user and may use these model(s) for subsequent prediction for that user.

Likewise, in some alternative implementations: (a) an initially trained model (e.g., a model trained as described in Step 106 in FIG. 1A) is applied to a new person for whom the system does not have an initial expert-provided-depression-rating or physiology-self-rating dating, by (i) in parallel hypothesizing with multiple (e.g., 20) different start-points of the model's baseline depression score, (ii) running the multiple versions in parallel to predict the person's next label(s), (iii) then asking a person how the person is doing or feeling and comparing what the person says to the different underlying models that were just ran; and (iv) then using the model that works best for that person.

This invention is not limited to ensemble machine learning models or to the machine learning models mentioned in FIGS. 2A and 2B. This invention may be implemented with any machine learning algorithm that performs well on the data.

This invention is not limited to cross-validation or to any particular split of training and test data (e.g., the 90% /10% splits mentioned in in FIGS. 2A and 2B). This invention may employ any method to validate data and may, if it splits data, split the data in any way.

Each smartphone in FIG. 4, and each other smartphone mentioned above, may comprise: (a) a smartphone; (b) a smartwatch; (c) smartglasses; (d) any other device that is configured to perform the communication functions of a smartphone; or (e) any other device that has a mobile operating system and that is configured to send or receive wireless communications.

### Software

In the Computer Program Listing above, 23 computer program files are listed. These 23 computer program files comprise software employed in a prototype implementation of this invention. To run these as Python™ software files, the filename extension for each would be changed from “.txt” to “.py”.

In the prototype, the dimensionality reduction.txt program may be employed for dimensionality reduction.

In the prototype, the following programs may be employed for feature generation: app_features.txt; call_features.txt; combine_features.txt; display_features.txt; EDA_features.txt; eda_features_calculation.txt; EDA_motionless_features.txt; HRV_features .txt; location_features.txt; location_smart_features.txt; motion_features.txt; sleep_features.txt; sms_features.txt; survey_features.txt; and survey_label_individualization.txt.

In the prototype, the HDRS_imputation_survey.txt program may be employed for HDRS_imputation.

In the prototype, the following programs may be employed for HDRS prediction: basic.txt; boosting.txt; ensemble.txt; gp.txt; rf.txt; and robust.txt.

In the prototype, one or more of these 23 software files may call on one or more of the following software libraries: biosppy (for EDA analysis); cvxEDA (for calculating features from EDA); sklearn (for machine learning models and feature transformations); numpy (for numerical calculations); pandas (for easier access and applying functions to data frames); geopy (to easily calculate circular distance between location data points); spicy (for conducting statistical analyses and signal processing); and matplotlib (for plotting graphs such time-series, bar charts, etc.)

This invention is not limited to the 23 computer program files listed in the Computer Program Listing above. Other software may be employed. Other software libraries may be called or may be called in different ways. Depending on the particular implementation, the software used in this invention may vary.

### Computers

In illustrative implementations of this invention, one or more computers (e.g., servers, network hosts, client computers, integrated circuits, microcontrollers, controllers, field-programmable-gate arrays, personal computers, digital computers, driver circuits, or analog computers) are programmed or specially adapted to perform one or more of the following tasks: (1) to control the operation of, or interface with, hardware components of an automated system for assessing depression, including any smartphone or wearable sensor module; (2) to extract features from a dataset; (3) to perform dimensionality reduction; (4) to perform one or more ensemble machine learning programs; (5) to impute depression ratings based on patient self-reports; (6) to predict a depression rating for a patient (e.g., based on passive sensor data and on a single depression rating by a clinician); (7) to receive data from, control, or interface with one or more sensors; (8) to perform any other calculation, computation, program, algorithm, or computer function described or implied herein; (9) to receive signals indicative of human input; (10) to output signals for controlling transducers for outputting information in human perceivable format; (11) to process data, to perform computations, and to execute any algorithm or software; and (12) to control the read or write of data to and from memory devices (tasks 1-12 of this sentence referred to herein as the “Computer Tasks”). The one or more computers may include computers 421, 422, 423, 424, 425, 426, 441, 442, 443, 444 and may also include computers housed in smartphones, cellphones or mobile phones (e.g. housed in smartphones 431, 432, 433, 451, 452, 453). The one or more computers may, in some cases, communicate with each other or with other devices: (a) wirelessly, (b) by wired connection, (c) by fiber-optic link, or (d) by a combination of wired, wireless or fiber optic links.

In exemplary implementations, one or more computers are programmed to perform any and all calculations, computations, programs, algorithms, computer functions and computer tasks described or implied herein. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium may comprise a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, computer-readable media. For example, in some cases, these instructions comprise instructions for a computer to perform any calculation, computation, program, algorithm, or computer function described or implied herein. For example, in some cases, instructions encoded in a tangible, non-transitory, computer-accessible medium comprise instructions for a computer to perform the Computer Tasks.

### Network Communication

In illustrative implementations of this invention, electronic devices (e.g., 421, 422, 423, 424, 425, 426, 441, 442, 443, 444, 431, 432, 433, 451, 452, 453) are each configured for wireless or wired communication with other devices in one or more networks.

For example, in some cases, one or more of these electronic devices each include a wireless module for wireless communication with other devices in a network. Each wireless module may include (a) one or more antennas, (b) one or more wireless transceivers, transmitters or receivers, and (c) signal processing circuitry. Each wireless module may receive and transmit data in accordance with one or more wireless standards.

In some cases, one or more of the following hardware components are used for network communication: a computer bus, a computer port, network connection, network interface device, host adapter, wireless module, wireless card, signal processor, modem, router, cables or wiring.

In some cases, one or more computers are programmed for communication over one or more networks. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), wireless standard (including IEEE 802.11 (wi-fi), IEEE 802.15 (bluetooth/zigbee), IEEE 802.16, IEEE 802.20 and including any mobile phone standard, including GSM (global system for mobile communications), UMTS (universal mobile telecommunication system), CDMA (code division multiple access, including IS-95, IS-2000, and WCDMA), or LTE (long term evolution)), or other IEEE communication standard.

### Definitions

The terms “a” and “an”, when modifying a noun, do not imply that only one of the noun exists. For example, a statement that “an apple is hanging from a branch”: (i) does not imply that only one apple is hanging from the branch; (ii) is true if one apple is hanging from the branch; and (iii) is true if multiple apples are hanging from the branch.

To compute “based on” specified data means to perform a computation that takes the specified data as an input.

The term “comprise” (and grammatical variations thereof) shall be construed as if followed by “without limitation”. If A comprises B, then A includes B and may include other things.

A human is not a “computer”, as that term is used herein.

“Defined Term” means a term or phrase that is set forth in quotation marks in this Definitions section.

“Depression rating” means a rating on a scale of levels of depression, which scale may also include at least one level for a state of being not depressed.

Depression rating “at” a time means a depression rating that rates level of depression, which level occurred at the time.

Depression rating “by” a person means a depression rating which is assessed, made, or assigned by the person.

Depression rating “for” a person means a depression rating that rates a level of depression of that person.

A “single” depression rating by a human means a depression rating by the human that is based solely on observations by, or interactions with, the human during a single period of time, which period is not longer than a day.

For an event to occur “during” a time period, it is not necessary that the event occur throughout the entire time period. For example, an event that occurs during only a portion of a given time period occurs “during” the given time period.

“EDA” means electrodermal activity.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each may be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

“For instance” means for example.

To say a “given” X is simply a way of identifying the X, such that the X may be referred to later with specificity. To say a “given” X does not create any implication regarding X. For example, to say a “given” X does not create any implication that X is a gift, assumption, or known fact.

“GPS” means global positioning system.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

“Input device” means a keyboard, computer mouse, touch screen, microphone or camera.

“Left forearm EDA” means EDA on a left forearm.

Unless the context clearly indicates otherwise, “or” means and/or. For example, A or B is true if A is true, or B is true, or both A and B are true. Also, for example, a calculation of A or B means a calculation of A, or a calculation of B, or a calculation of A and B.

Measurement “of” a person means a measurement that records a physical aspect of the person, and does not mean a measurement taken by the person. For instance, the physical aspect may be EDA, temperature, heart pulse rate, heart rate variability, posture, facial expression, motion, or activity level.

To take measurements “over time” means to take measurements at multiple different times or during multiple different time intervals.

“Motion data” means data regarding motion.

Usage “over time” means usage that occurs at multiple different times or during multiple different time periods.

A parenthesis is simply to make text easier to read, by indicating a grouping of words. A parenthesis does not mean that the parenthetical material is optional or may be ignored.

“Physiological sensor measurement” means a measurement of a physical aspect of a person. For instance, the physical aspect may be EDA, temperature, heart pulse rate, heart rate variability, posture, facial expression, motion, or activity level.

“Right forearm EDA” means EDA on a right forearm.

“Self-report” means a dataset that encodes one or more subjective assessments by a person, which one or more assessments include at least one assessment that is indicative of affect of the person.

“SMS” means short message service.

As used herein, the term “set” does not include a group with no elements.

Unless the context clearly indicates otherwise, “some” means one or more.

“Smartphone” means a device that is configured for cellular phone communications and configured for accessing data external to the device and for audibly or visually presenting information to a user. Each of the following is a non-limiting examples of a smartphone:(a) an Apple® iPhone®; (b) a device that has an Android® operating system and is configured for cellular phone communications and for accessing data external to the device and for audibly or visually presenting information to a user; (c) a smartwatch that satisfies the requirements of the first sentence of this paragraph; and (d) smartglasses that satisfy the requirements of the first sentence of this paragraph is a “smartphone”.

As used herein, a “subset” of a set consists of less than all of the elements of the set.

The term “such as” means for example.

“Temperature data” means data regarding temperature.

To say that a machine-readable medium is “transitory” means that the medium is a transitory signal, such as an electromagnetic wave.

“User” means a human user.

Except to the extent that the context clearly requires otherwise, if steps in a method are described herein, then the method includes variations in which: (1) steps in the method occur in any order or sequence, including any order or sequence different than that described herein; (2) any step or steps in the method occur more than once; (3) any two steps occur the same number of times or a different number of times during the method; (4) any combination of steps in the method is done in parallel or serially; (5) any step in the method is performed iteratively; (6) a given step in the method is applied to the same thing each time that the given step occurs or is applied to different things each time that the given step occurs; (7) one or more steps occur simultaneously, or (8) the method includes other steps, in addition to the steps described herein.

Headings are included herein merely to facilitate a reader's navigation of this document. A heading for a section does not affect the meaning or scope of that section.

This Definitions section shall, in all cases, control over and override any other definition of the Defined Terms. The Applicant or Applicants are acting as his, her, its or their own lexicographer with respect to the Defined Terms. For example, the definitions of Defined Terms set forth in this Definitions section override common usage and any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. Unless the context clearly indicates otherwise, any definition or clarification herein of a term or phrase applies to any grammatical variation of the term or phrase, taking into account the difference in grammatical form. For example, the grammatical variations include noun, verb, participle, adjective, and possessive forms, and different declensions, and different tenses.

### Variations

This invention may be implemented in many different ways. Here are some non-limiting examples:

In some implementations, this invention is a method comprising: (a) accepting, as input, a first set of depression ratings by one or more humans, in such a way that the first set of depression ratings includes, for each specific patient in a set of multiple patients, a depression rating for the specific patient at each time in a first set of times during a training period; (b) accepting, as input, self-reports by each of the patients over time during the training period; (c) taking a first set of physiological sensor measurements of each of the patients over time during the training period; (d) accepting, as input, data regarding smartphone usage or short message service (SMS) usage of each of the patients over time during the training period; (e) estimating, based on the self-reports, a second set of depression ratings, in such a way that (i) the second set of depression ratings includes, for each specific patient in the set of patients, a depression rating for the specific patient at each time in a second set of times during the training period, and (ii) the first set of times and the second set of times are non-overlapping; (f) training a machine learning model on a training dataset, in such a way that (i) the training results in a trained machine learning model, and (ii) the training dataset comprises the first set of depression ratings, the second set of depression ratings, the self-reports and the first set of data regarding smartphone usage or SMS usage; (g) after the training period (i) accepting, as input, an additional depression rating for a user, which additional rating is by a human and occurs at a specific time in an evaluation period, and (ii) taking a second set of physiological sensor measurements, which second set of physiological measurements are of the user over time during the evaluation period; (iii) accepting, as input, a second set of data regarding smartphone usage or SMS usage, which second set of data comprises data regarding smartphone usage or SMS usage of the user over time during the evaluation period; and (h) performing calculations to determine a depression rating for the user at one or more times that are in the evaluation period and are different than the specific time, which calculations are by the trained machine learning model and are based on (A) the additional depression rating, (B) the second set of physiological sensor measurements, and (C) the second set of data regarding smartphone usage or SMS usage. In some cases, the estimating of the second set of depression ratings is by an ensemble machine learning model. In some cases, the trained machine learning model is an ensemble machine learning model. In some cases, the method further comprises performing dimensionality reduction on the self-reports. In some cases, the additional depression rating and each depression rating in the first and second sets of depression ratings is a rating on a Hamilton Depression Rating Scale. In some cases, the calculations to determine a depression rating for the user are not based on self-reports by the user. In some cases: (a) the first and second sets of physiological sensor measurements each include measurements of electrodermal activity (EDA); and (b) the method further comprises computing a measure of asymmetry between right forearm EDA and left forearm EDA. In some cases: (a) the first and second sets of physiological measurements each include measurements of electrodermal activity (EDA) and of temperature; and (b) EDA measurements taken when temperature is below a specified threshold are not employed for calculating depression ratings. In some cases, the first and second sets of physiological sensor measurements each include accelerometer data. In some cases: (a) the first and second sets of physiological sensor measurements each include motion data or temperature data; and (b) the method further comprises detecting a sleep state or a level of activity based on the motion data or temperature data. In some cases, the first and second sets of smartphone usage data each include data regarding whether a smartphone display is on or off. In some cases, the first and second sets of smartphone usage data each include data regarding duration of a time interval in which a smartphone display is on. In some cases, the first and second sets of smartphone usage data each include metadata regarding phone calls. In some cases, the first and second sets of SMS usage data comprises data regarding frequency or number of incoming or outgoing SMS messages. Each of the cases described above in this paragraph is an example of the method described in the first sentence of this paragraph, and is also an example of an embodiment of this invention that may be combined with other embodiments of this invention.

In some implementations, this invention is a method comprising: (a) accepting, as input, a depression rating for a user, which depression rating is by a human and occurs at a specific time during a temporal period; (b) taking physiological sensor measurements, which physiological measurements are of the user over time during the period; (c) accepting, as input, data regarding smartphone usage or SMS usage of the user over time during the period; and (d) performing calculations to determine a depression rating for the user at one or more times that are in the period and are different than the specific time, which calculations are by a trained machine learning model and are based on (i) the depression rating, (ii) the physiological sensor measurements, and (iii) the data regarding smartphone usage or SMS usage. In some cases, the calculations to determine a depression rating are not based on self-reports by the user. In some cases, the trained machine learning model is an ensemble machine learning model. In some cases: (a) the physiological sensor measurements include measurements of electrodermal activity (EDA) on a right forearm of the user and on a left forearm of the user; and (b) the method further comprises computing a measure of asymmetry between the EDA on the right forearm and the EDA on the left forearm. Each of the cases described above in this paragraph is an example of the method described in the first sentence of this paragraph, and is also an example of an embodiment of this invention that may be combined with other embodiments of this invention.

In some implementations, this invention is a system comprising: (a) one or more sensors; and (b) one or more computers; wherein (i) the one or more sensors are configured to take physiological sensor measurements of a user over time during a temporal period, and (ii) the one or more computers are programmed (A) to accept, as input, a depression rating for a user, which depression rating is by a human and occurs at a specific time in the period, (B) to accept, as input, data regarding smartphone usage or SMS usage of the user over time during the period, and (C) to perform calculations to determine a depression rating for the user at one or more times that are in the period and are different than the specific time, which calculations are by a trained machine learning model and are based on (I) the depression rating, (II) the physiological sensor measurements, and (III) the data regarding smartphone usage or SMS usage. In some cases, the calculations to determine a depression rating are not based on self-reports by the user. Each of the cases described above in this paragraph is an example of the system described in the first sentence of this paragraph, and is also an example of an embodiment of this invention that may be combined with other embodiments of this invention.

Each description herein (or in the Provisional) of any method, apparatus or system of this invention describes a non-limiting example of this invention. This invention is not limited to those examples, and may be implemented in other ways.

Each description herein (or in the Provisional) of any prototype of this invention describes a non-limiting example of this invention. This invention is not limited to those examples, and may be implemented in other ways.

Each description herein (or in the Provisional) of any implementation, embodiment or case of this invention (or any use scenario for this invention) describes a non-limiting example of this invention. This invention is not limited to those examples, and may be implemented in other ways.

Each Figure herein (or in the Provisional) that illustrates any feature of this invention shows a non-limiting example of this invention. This invention is not limited to those examples, and may be implemented in other ways.

The above description (including without limitation any attached drawings and figures) describes illustrative implementations of the invention. However, the invention may be implemented in other ways. The methods and apparatus which are described herein are merely illustrative applications of the principles of the invention. Other arrangements, methods, modifications, and substitutions by one of ordinary skill in the art are also within the scope of the present invention. Numerous modifications may be made by those skilled in the art without departing from the scope of the invention. Also, this invention includes without limitation each combination and permutation of one or more of the items (including hardware, hardware components, methods, processes, steps, software, algorithms, features, or technology) that are described herein.

What is claimed:

1. A method comprising:
(a) accepting, as input, a first set of depression ratings by one or more humans, in such a way that the first set of depression ratings includes, for each specific patient in a set of multiple patients, a depression rating for the specific patient at each time in a first set of times during a training period;
(b) accepting, as input, self-reports by each of the patients over time during the training period;
(c) taking a first set of physiological sensor measurements of each of the patients over time during the training period;
(d) accepting, as input, data regarding smartphone usage or short message service (SMS) usage of each of the patients over time during the training period;
(e) estimating, based on the self-reports, a second set of depression ratings, in such a way that (i) the second set of depression ratings includes, for each specific patient in the set of patients, a depression rating for the specific patient at each time in a second set of times during the training period, and (ii) the first set of times and the second set of times are non-overlapping;
(f) training a machine learning model on a training dataset, in such a way that (i) the training results in a trained machine learning model, and (ii) the training dataset comprises the first set of depression ratings, the second set of depression ratings, the self-reports and the first set of data regarding smartphone usage or SMS usage;
(g) after the training period
(i) accepting, as input, an additional depression rating for a user, which additional rating is by a human and occurs at a specific time in an evaluation period, and
(ii) taking a second set of physiological sensor measurements, which second set of physiological measurements are of the user over time during the evaluation period;
(iii) accepting, as input, a second set of data regarding smartphone usage or SMS usage, which second set of data comprises data regarding smartphone usage or SMS usage of the user over time during the evaluation period; and
(h) performing calculations to determine a depression rating for the user at one or more times that are in the evaluation period and are different than the specific time, which calculations are by the trained machine learning model and are based on (A) the additional depression rating, (B) the second set of physiological sensor measurements, and (C) the second set of data regarding smartphone usage or SMS usage.

2. The method of claim 1, wherein the estimating of the second set of depression ratings is by an ensemble machine learning model.

3. The method of claim 1, wherein the trained machine learning model is an ensemble machine learning model.

4. The method of claim 1, wherein the method further comprises performing dimensionality reduction on the self-reports.

5. The method of claim 1, wherein the additional depression rating and each depression rating in the first and second sets of depression ratings is a rating on a Hamilton Depression Rating Scale.

6. The method of claim 1, wherein the calculations to determine a depression rating for the user are not based on self-reports by the user.

7. The method of claim 1, wherein:
(a) the first and second sets of physiological sensor measurements each include measurements of electrodermal activity (EDA); and
(b) the method further comprises computing a measure of asymmetry between right forearm EDA and left forearm EDA.

8. The method of claim 1, wherein:
(a) the first and second sets of physiological measurements each include measurements of electrodermal activity (EDA) and of temperature; and
(b) EDA measurements taken when temperature is below a specified threshold are not employed for calculating depression ratings.

9. The method of claim 1, wherein the first and second sets of physiological sensor measurements each include accelerometer data.

10. The method of claim 1, wherein:
(a) the first and second sets of physiological sensor measurements each include motion data or temperature data; and
(b) the method further comprises detecting a sleep state or a level of activity based on the motion data or temperature data.

11. The method of claim 1, wherein the first and second sets of smartphone usage data each include data regarding whether a smartphone display is on or off

12. The method of claim 1, wherein the first and second sets of smartphone usage data each include data regarding duration of a time interval in which a smartphone display is on.

13. The method of claim 1, wherein the first and second sets of smartphone usage data each include metadata regarding phone calls.

14. The method of claim 1, wherein the first and second sets of SMS usage data comprises data regarding frequency or number of incoming or outgoing SMS messages.

15. A method comprising:
(a) accepting, as input, a depression rating for a user, which depression rating is by a human and occurs at a specific time during a temporal period;
(b) taking physiological sensor measurements, which physiological measurements are of the user over time during the period;
(c) accepting, as input, data regarding smartphone usage or SMS usage of the user over time during the period; and
(d) performing calculations to determine a depression rating for the user at one or more times that are in the period and are different than the specific time, which calculations are by a trained machine learning model and are based on (i) the depression rating, (ii) the physiological sensor measurements, and (iii) the data regarding smartphone usage or SMS usage.

16. The method of claim 15, wherein the calculations to determine a depression rating are not based on self-reports by the user.

17. The method of claim 15, wherein the trained machine learning model is an ensemble machine learning model.

18. The method of claim 15, wherein:
(a) the physiological sensor measurements include measurements of electrodermal activity (EDA) on a right forearm of the user and on a left forearm of the user; and
(b) the method further comprises computing a measure of asymmetry between the EDA on the right forearm and the EDA on the left forearm.

19. A system comprising:
(a) one or more sensors; and
(b) one or more computers;
wherein
(i) the one or more sensors are configured to take physiological sensor measurements of a user over time during a temporal period, and
(ii) the one or more computers are programmed
(A) to accept, as input, a depression rating for a user, which depression rating is by a human and occurs at a specific time in the period,
(B) to accept, as input, data regarding smartphone usage or SMS usage of the user over time during the period, and
(C) to perform calculations to determine a depression rating for the user at one or more times that are in the period and are different than the specific time, which calculations are by a trained machine learning model and are based on (I) the depression rating, (II) the physiological sensor measurements, and (III) the data regarding smartphone usage or SMS usage.

20. The system of claim 19, wherein the calculations to determine a depression rating are not based on self-reports by the user.
